(19) United States
(12) Patent Application Publication
(10) Pub. No.: US 2025/0325305 A1
(43) Pub. Date: Oct. 23, 2025

(54) VERTEBRAL STABILIZATION DEVICE

(71) Applicant: Paul FAYADA, Mauzac (FR)

(72) Inventor: Paul FAYADA, Mauzac (FR)

(21) Appl. No.: 18/870,031

(22) PCT Filed: May 30, 2023
(86) PCT No.: PCT/EP2023/064459

§ 371 Date: Nov. 27, 2024

(30) Foreign Application Priority Data
Jun. 1, 2022 (FR) FR2205293

(51) Int. Cl. A61B 17/70

(52) U.S. Cl. CPC A61B 17/7011; A61B 17/7008; A61B 17/7035

(57) ABSTRACT

The invention relates to a vertebral stabilization device comprising at least two screws, each having a first portion engaged in a vertebra and a second portion separated from the first by a flange. The device further comprises at least a first connection rod for connecting the screws, and at least a first securing assembly and a second securing assembly for securing the rod to the screws. Each securing assembly comprises at least one clip, which is able to be engaged on the second portion of one of the screws, said clip being arranged to clamp one end of the rod, and it comprises a nut designed to be screwed onto the second portion and clamp the clip directly or indirectly against the flange of the screw. Each securing assembly is designed to create at least one ball-and-socket joint connection between the clip and the second portion of the screw.

### BACKGROUND OF THE INVENTION

The present invention relates to the field of surgical implants and more specifically, vertebral stabilization devices implanted in a human or animal body for the treatment of pathologies of the spine.

There are numerous vertebral stabilization devices, the general principle of which is to connect adjacent vertebrae by rods.

In a first device, the rods (sometimes called bars) are secured on either side of the vertebrae to be connected along a plane substantially parallel to the median sagittal plane of the vertebral column. This device makes it possible to obtain a very good stabilization of the vertebrae against one another, but has the main disadvantage of significantly limiting the mobility of the column, impeding, even preventing certain movements of the patient. The rods are directly inserted into the pedicle screw heads, which involves the screw heads being perfectly aligned against one another, which is sometimes difficult to obtain.

In a second device forming the subject of the patent application WO 2018/019792, the rods are secured, such that the rods cross one another over the median sagittal plane of the vertebral column. This latter device makes it possible to preserve a certain mobility between the two vertebrae connected by the device, while reinforcing their stability. The rods have a first circular ring-shaped end and a second oblong ring-shaped end. During the implementation, the rings are engaged respectively on threaded portions extending the pedicle screws, screwed directly into the vertebrae. The definitive securing of the vertebrae is done by screwing a nut onto each threaded portion to block the ring of the rod by clamping, thus securing the position of the rod with respect to the pedicle screws. This securing principle, although simple, is quite difficult to adjust accurately. Furthermore, during the clamping of the nuts, there can be a slight movement of the rod around the securing elements, modifying the initial adjustment performed by the surgeon. Thus, the implementation of this device requires great control and the surgeon being highly meticulous, which generally has the consequence of extending the duration of interventions.

### AIM OF THE INVENTION

The invention aims, in particular, to overcome at least partially, the abovementioned disadvantages, by proposing a vertebral stabilization device, which is compact and the implantation of which is easy and rapid.

### SUMMARY OF THE INVENTION

To this end, a vertebral stabilization device is provided, comprising at least two screws, each having a first threaded portion to be engaged in a vertebra and a second threaded portion separated from the first threaded portion by a flange. The device of the invention further comprises at least one first connection rod having two opposite ends to connect one to the other of the two threaded portions of the screws, and at least one first securing assembly and one second securing assembly to secure the ends of the rod to the screws. Each securing assembly of the device which is the subject matter of the invention, comprises at least one clip, which is able to be engaged on the second threaded portion of one of the screws, said clip being arranged to clamp one end of the rod, and comprises a nut which has a lower bearing surface, said nut is designed to be screwed on the second threaded portion and clamp the clip directly or indirectly against an upper surface of the flange of the screw. Each securing assembly is designed to create at least one ball-and-socket joint connection between the clip and the second threaded portion.

Thus, according to the invention, the surgeon can perform the stabilization of at least two vertebrae by using at least one stabilization device according to the invention. The stabilization of the vertebrae is easily performed by way of rods simply secured by the clips of the securing assemblies, equally crossed over a median sagittal plane of the vertebral column or according to a conventional parallel mounting in a plane parallel to the median sagittal plane of the column. The device according to the invention enables not only an easier and more reliable securing of the rods, but also resume mountings in patients operated on, with, for example, an extension of the mounting, without having to dismount the assembly as is necessarily the case with the currently available instrumentations.

Preferably, the lower bearing surface of the nut and the upper bearing surface of the flange are spherical cap-shaped concave surfaces, identical to one another.

According to a first particular arrangement according to the invention, the clip at least of the first securing assembly comprises a first jaw having a spherical cap-shaped convex outer surface to bear, in this case directly, on the upper surface of the flange and a second jaw having a spherical cap-shaped convex outer surface, the first securing assembly comprising a first support sleeve for the nut, said first support sleeve having a complementary concave lower front surface to bear against the spherical cap-shaped convex outer surface of the second jaw. The first support sleeve can be distinct from the nut. Particularly, the first support sleeve comprises a spherical cap-shaped convex upper front surface complementary of the lower surface of the nut to bear against it.

According to a second particular arrangement according to the invention, the clip at least of the second securing assembly comprises a first jaw having a complementary spherical cap-shaped convex outer surface to bear against the lower surface of the nut and a third jaw having a spherical cap-shaped concave outer surface. The second securing assembly further comprises a second support sleeve having a complementary upper front surface to bear on the spherical cap-shaped concave outer surface of the third jaw. Particularly, the second support sleeve has a spherical cap-shaped concave lower front surface to bear on the upper surface of the flange. In this case, the nut makes it possible to clamp the clip indirectly against the upper surface of the flange of the screw, indeed according to this second arrangement, the second support sleeve is between the flange and the clip.

The presence of a support sleeve in the first or the second arrangement thus makes it possible to adjust the height of the securing assembly on the second threaded portion of the screw thus enabling a positioning of the rods adapted to the morphology of the patient and to the pathology to be treated. In particular, when the vertebral stabilization is performed by crossing rods about the median sagittal axis, the support sleeves make it possible to adjust the height of the rods against one another without friction, or with a controlled contact.

According to a third arrangement according to the invention, the second securing assembly comprises two clips, namely a lower clip and an upper clip. The lower clip comprises a first jaw having a complementary spherical cap-shaped convex outer surface to bear directly on the upper surface of the flange and a second jaw having a spherical cap-shaped convex outer surface. The upper clip comprises a first jaw having a complementary spherical cap-shaped convex outer surface to bear against the lower surface of the nut and a third jaw having a spherical cap-shaped concave outer surface complementary of the convex outer surface of the second jaw of the first clip, the concave outer surface of the third jaw of the second clip bearing on the convex outer surface of the second jaw of the first clip.

According to the invention, each clip comprises an elastically deformable portion connecting the jaws to one another, such that these are movable against one another between an open position and a closed position.

According to a favoured embodiment of the invention, the first jaw is provided with a bore to be engaged in the second threaded portion of the screw. The bore comprises a first cone frustum-shaped portion and a second cylindrical portion. The first portion has a large cross-section which opens onto the outer surface of the first jaw and a small cross-section combined with a cylindrical cross-section of the second portion, the second portion opening onto the internal surface of the first jaw.

The second jaw is also provided with a bore to be engaged on the second threaded portion of the screw. The bore comprises, in this case, a first truncated cone-shaped portion and a second truncated cone-shaped portion, the first portion having a large cross-section opening onto the outer surface of the second jaw and a small cross-section combined with a small cross-section of the second portion. The second portion has a large cross-section which opens onto the inner surface of the second jaw. The common centre of rotation of the surfaces in contact with the nut and the flange is disposed at the centre of the small cross-sections of the first and second portions. Symbolically, the bore has a shape similar to that of a diabolo.

Optionally, the flange of the screw can be removable.

The particular bore of the clips, the shape of the outer surfaces of the clips, as well as that of the outer surface of the flange, and the lower surface of the nut, just like that of the upper and lower front surfaces of the support sleeves make it possible to perform the ball-and-socket joint connection between the different elements of the device (flange, clip(s), support sleeve(s), nut), which makes it possible for the surgeon to extremely accurately and easily adjust the positioning of the rods according to the morphology of the patient and to the pathology to be treated. Once the optimal positioning is obtained, the latter is blocked by simple clamping of the nut on each of the screws.

According to a particular embodiment of the invention, the rod has a constant cross-section over its entire length. Preferably, the rod can be curved. The word “rod” means, in this case, the entire part, extended and relatively narrow with respect to its length, whatever its cross-section, the rods of which according to the common meaning of this word and meaning of bars.

Preferably, the second threaded portion of each screw is provided with an end portion arranged to enable a rotating connection with a tool for screwing the screw into the vertebra. It also has a breaking piece to be able to separate the end portion from the second threaded portion by breaking.

Thus, at the end of the intervention, after the screws have been screwed into the vertebrae and after the device according to the invention has been put in place, the surgeon can shorten each of the screws at the breaking piece to make the device more compact and more comfortable for the patient.

Other features and advantages of the invention will emerge upon reading the description below of a particular and non-limiting embodiment of the invention.

### BRIEF DESCRIPTION OF THE DRAWINGS

Reference will be made to the accompanying drawings, among which:

FIG. 1: schematic view of three vertebrae as a top view and in a slight perspective, equipped with a device according to a particular embodiment of the invention;

FIG. 2: schematic view in a longitudinal cross-section of one of the screws of this device, provided with a securing assembly according to a first configuration comprising a clip, a support sleeve and a nut;

FIG. 3: schematic view in a longitudinal cross-section of one of the screws of this device, provided with a securing assembly according to a second configuration comprising a support sleeve, a clip, and a nut;

FIG. 4: schematic view in perspective of a screw this device, provided with a securing assembly according to a third configuration comprising a first clip, a second clip and a nut;

FIG. 5: schematic view of this screw in a longitudinal cross-section along the plane V of FIG. 4;

FIG. 6: schematic view of this screw in a longitudinal cross-section along the plane VI of FIG. 4 of a securing assembly inclined by ball-and-socket joint connection with respect to the screw;

FIG. 7: exploded, schematic view of this screw and of the securing assembly;

FIG. 8: exploded, schematic view, in a longitudinal cross-section along the plane V of FIG. 4, of this screw and of the securing assembly;

FIG. 9: perspective, schematic view of a pin of a clip.

### DETAILED DESCRIPTION OF THE INVENTION

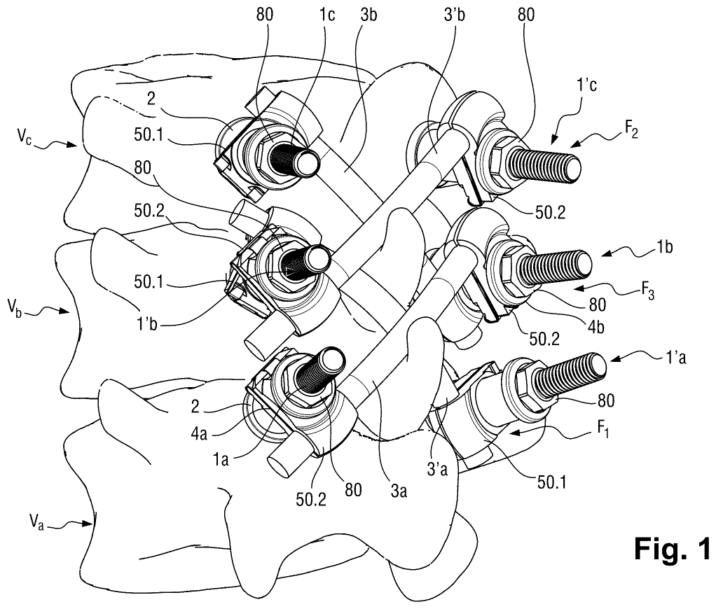

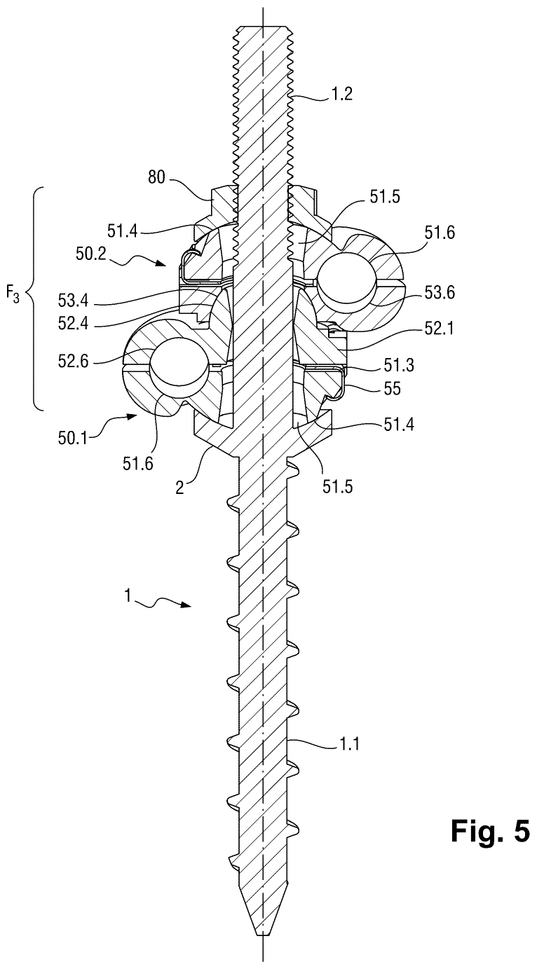

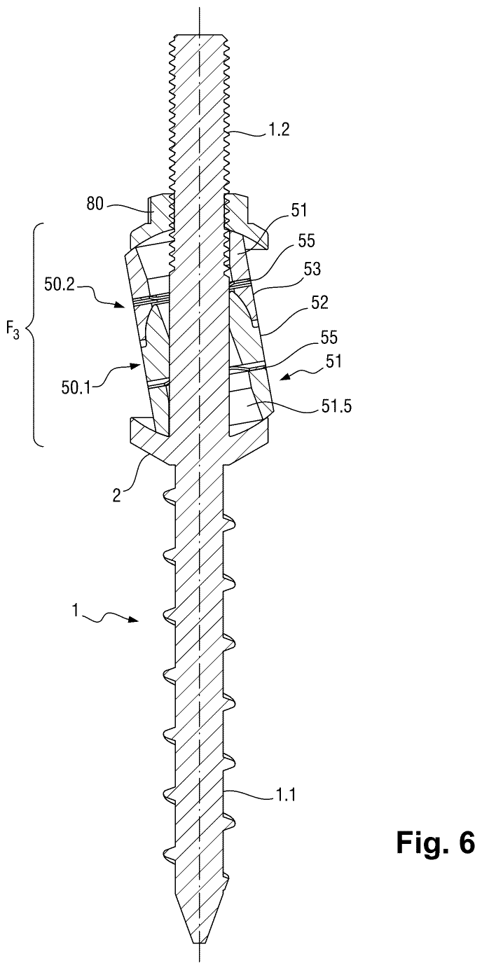

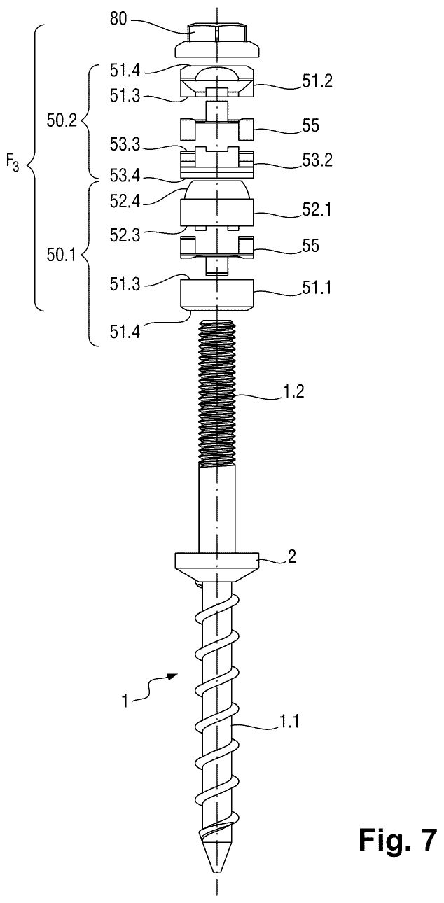

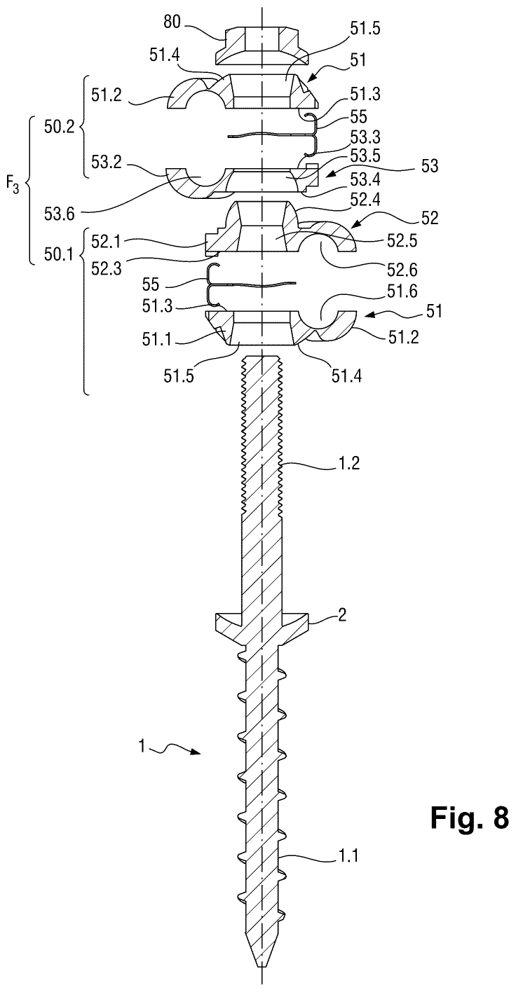

In reference to FIGS. 1 to 9, the present invention relates to a vertebral stabilization device, comprising:
at least two screws 1a; 1b; each provided with a flange 2;
at least one first connection rod 3;
at least one first securing assembly and one second securing assembly for securing the ends of the rod 3 respectively to the screws 1a and 1b. Each securing assembly comprises at least one clip 50.1, 50.2, which is able to be engaged on the second threaded portion 1.2 of one of the screws and designed to clamp an end of the rod 3, and a nut 80.

According to an example of apparatus of a vertebral column represented in FIG. 1, three adjacent vertebrae Va, Vb, Vc are stabilized with a vertebral stabilization device according to the invention.

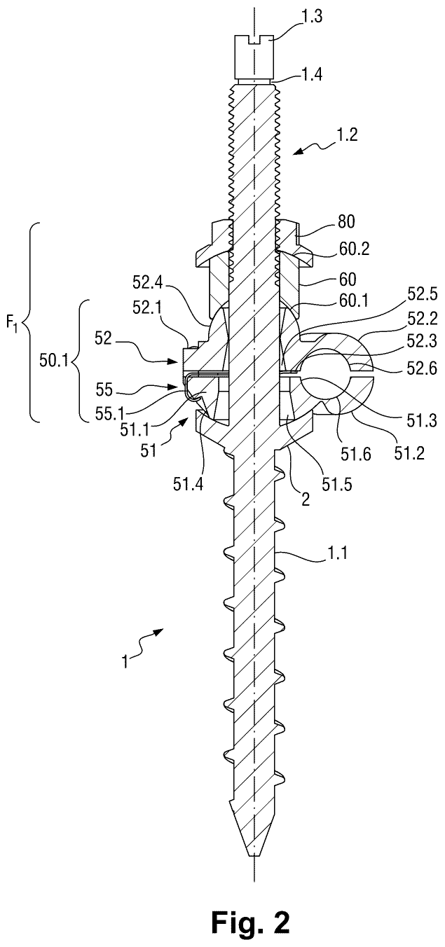

In this case, the device comprises two screws referenced 1a, 1′a on the vertebra Va, two screws 1b, 1′b on the vertebra Vb and two screws 1c, 1′c on the vertebra Vc. All the screws 1, 1′ are identical, the letters a, b, c only serving to distinguish the screws according to the vertebra on which they are implanted. Each screw thus comprises a first threaded portion 1.1, not represented in FIG. 1, but which can be seen in FIGS. 2 to 7, to be engaged in the vertebra and a second threaded portion 1.2 separated from the first threaded portion 1.1 by a flange 2.

The vertebrae Va, Vb, Vc to be instrumented are generally prepared beforehand before receiving the stabilization device. Conventionally, the articular or spinous processes of the vertebrae can have been reduced beforehand, and the vertebrae drilled beforehand to receive the screws.

As indicated above, the first threaded portion 1a.1, 1b.1, 1c.1 of the screws 1a, 1b, 1c are inserted in a staggered manner into the vertebrae Va, Vb and Vc on either side of the median sagittal plane and alternatively. Thus, the first portion 1a.1 of the screw 1a is inserted into the vertebra Va to the left of the median sagittal plane, the first portion 1b.1 of the screw 1b is inserted into the vertebra Vb to the right of the median sagittal plane and the first portion 1c.1 of the screw 1c is inserted into the vertebra Vc to the left of the median sagittal plane. Symmetrically, the first portion 1′a.1 of the screw 1′a is inserted into the vertebra Va to the right of the median sagittel plane more or less facing the screw 1a. In the same way, the first portion 1′b.1 of the screw 1′b is inserted into the vertebra Vb to the left of the median sagittal plane more or less facing the screw 1b and the first portion 1′c.1 of the screw 1′c is inserted into the vertebra Vc to the right of the median sagittal plane more or less facing the screw 1c. Thus, each vertebra Va, Vb, Vc is equipped with two screws respectively 1a and 1′a; 1b and 1′b; 1c and 1′c.

With all the screws being identical, the description below of a screw applies to all screws. As indicated above, each screw 1 comprises a first portion 1.1 and a second portion 1.2, separated by a flange 2. The first portion 1.1 is completely inserted into the bone of the vertebra up to the flange 2. The flange 2 is thus located on the surface of the vertebra, bearing on the bone, the second portion of the screw 1.2 projecting from the vertebra.

The surgeon knows how to drill and correctly position the screws 1 according to the morphology of the patient.

A person skilled in the art will know how to shape the first portion 1.1, so as to facilitate its engagement in the bone. For example, the first portion can be self-tapping and have an end shaped in the form of a tip. In a variant, the first portion can also be self-drilling.

The flange 2 which separates the first and the second portion 1.1; 1.2 of the screw 1 has a spherical cap-shaped convex lower surface in contact with the bone of the vertebra and a spherical cap-shaped concave bearing upper surface. A person skilled in the art will know how to adapt the diameter and the convexity of the flange according to the anatomy of the patient and of the mechanical stresses undergone.

The second portion 1.2 of the screw 1 thus projects from the vertebra when the screw 1 is secured. The threading of the second portion 1.2 is done, so as to receive a securing assembly F. Preferably, the second threaded portion 1.2 is provided with an end portion designed to enable a rotating connection with a screwing tool. The end portion, represented in FIG. 2 only, can thus comprise a footprint 1.3 shaped complementarily to that of the tip of a screwdriver. As non-limiting examples, the footprint can be hexagonal, cross-headed, starred or triangular. Furthermore, the second threaded portion 1.2 can comprise a breaking piece 1.4 to be able to separate the end portion from the second threaded portion by breaking, when the surgical intervention has ended.

A person skilled in the art will know how to manufacture the screw, in this case made of one piece and contiguous between the first screw portion, the second screw portion and the flange, made of biocompatible and stainless material, sufficiently mechanically and chemically resistant for the application considered.

A person skilled in the art will know how to determine the diameter and the length of the screw to be the best adapted to the age, to the morphology and to the bone quality of the patient. As an illustration, the length of the screws can be conventionally between 65 mm and 90 mm.

According to this particular embodiment of the invention, the device each comprises two pairs of connection rods, respectively referenced 3a, 3b and 3′a, 3′b. Each rod has two opposite ends to connect the second threaded portions of two of the screws to one another and in pairs.

Thus, as illustrated in FIG. 1, a first connection rod 3a will connect the second portion 1a.2 of the screw 1a to the second portion 1b.2 of the screw 1b and a second connection rod 3b will connect the second portion 1b.2 of the screw 1b to the second portion 1c.2 of the screw 1c, such that the screws 1a, 1b, 1c are connected in pairs. In the same way, a third rod 3′a will connect the second portion 1′a.2 of the screw 1′a to the second portion 1′b.2 of the screw 1′b, and a fourth rod 3′b will connect the second portion 1′b.2 of the screw 1′b to the second portion 1′c.2 of the screw 1′c, such that the screws 1′a, 1′b, 1′c are connected in pairs. The rods 3a, 3′b are thus parallel to one another as the rods 3′a and 3b also are to one another. The rods 3a and 3′a stabilize the vertebrae Va and Vb to one another, the rods 3b and 3′b, the vertebrae Vb and Vc crossing one another over the median sagittal plane of the column respectively between the vertebrae Va and Vb and between the vertebrae Vb and Vc.

Each rod 3 is, in this case, made of biocompatible material, and has a constant cross-section over its entire length. The cross-section is circular, in this case.

The rods 3 can be made of any sufficiently rigid biocompatible material to obtain the stabilization of the vertebrae to one another. For example, a material having a Young's modulus of between 3.6 GPa and 60 GPa will be chosen. As an example, the material can be a composite material comprising a mixture of carbon and polyetheretherketone (PEEK) fibres.

A person skilled in the art is able to determine the diameter of the cross-section of the rod according to the material used, to the sought mechanical features and to the anatomy of the patient. They will also know how to determine the length of the rods to adapt it to the morphology and to the pathology of the patient and according to which the rods are placed parallel to the median sagittal plane or according to which they cross one another over it.

Preferably, and according to the particular embodiment of the invention, the rods are curved, thus facilitating their implementation and limiting the interferences with the central part of the coupled vertebrae.

The securing of each end of the rods 3 to one of the screws 1 is ensured by a securing assembly F engaged on the second threaded portion of the screw.

Each securing assembly F according to the invention comprises at least one clip 50.1, 50.2 which is able to be engaged on the second threaded portion 1.2 of one of the screws 1 and designed to clamp an end of the rod 3, and a nut 80 which has a lower bearing surface and which is designed to be screwed on the second threaded portion 1.2 by clamping the clip 50.1, 50.2 against the upper surface of the flange 2 of the screw 1. Each securing assembly F is designed to create a ball-and-socketjoint connection between the clip 50.1, 50.2 and the second threaded portion 1.2. Preferably, the lower bearing surface of the nut 80 and the upper bearing surface of the flange 2 such as described above are spherical cap-shaped concave surfaces identical to one another.

The securing assembly F can have several configurations, according to which it is located on a screw implanted in one of the end vertebrae of the part of the fitted column (vertebrae Va, Vc) or on a screw implanted in an intermediate vertebra (vertebra Vb). The securing assembly F will thus comprise a support sleeve in the two configurations adapted to the end vertebrae or a second clip in the configuration adapted to the intermediate vertebrae.

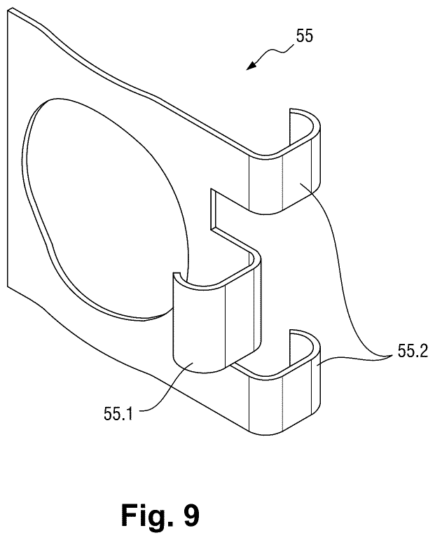

In reference to FIG. 2, the securing assembly F1 according to the first configuration comprises a clip 50.1, a support sleeve 60 and a nut 80. The clip 50.1 is, in this case, more or less rectangular in shape, and comprises two jaws 51, 52 connected by an elastically deformable portion, in this case, a metal spring pin 55, such that the jaws are movable against one another between an open position and a closed position. The pin 55 illustrated in FIG. 9 comprises a first hook-shaped tab 55.1 which elastically surrounds a portion of the first part 51.1 opposite the second part 51.2 and two hook-shaped tabs 55.2 which elastically surround a portion of the first part 52.1 opposite the second part 52.2. A person skilled in the art will know how to produce the elastically deformable portion differently. They can, for example, produce the clip in one single part, the two jaws being connected to one another by a one-piece elastically deformable portion. The two jaws can be connected to one another by a hinge, optionally associated with a spiral spring.

Each jaw 51 comprises a first part 51.1, or part for connecting to the screw 1, intended to be engaged on the second portion 1.2 of the screw 1, and a second part 51.2, or part for connecting the rod 3, which laterally projects with respect to the first part 51.1 and which is designed to receive one of the ends of one of the rods 3.

The first part 51.1 is, in this case, more or less parallelepiped in shape, with a square cross-section with a side which is substantially equal to or slightly greater than the diameter of the flange 2. The first part 51.1 comprises an inner surface 51.3 facing the jaw 52 (and therefore rotated inwards from the clip 50.1) and opposite an outer surface 51.4 rotated outwards from the clip 50.1. The inner surface 51.3 is a flat surface. The outer surface 51.4 is spherical cap-and convex-shaped complementarily to that of the upper surface of the flange 2 so as to be able to bear on the surface on it.

The first part 51.1 of the first jaw 51 is provided with a bore 51.5 to be engaged on the second threaded portion 1.2 of the screw 1. The bore 51.5 opens on one side onto the inner surface 51.3 of the first part 51.1 of the jaw perpendicularly to it, and on the other side, on the outer surface 51.4. The bore 51.5 comprises in this case, along a central axis, a first truncated cone-shaped portion and a second cylindrical portion, the first portion having a large cross-section opening onto the outer surface 51.4 and a small cross-section combined with a cylindrical cross-section of the second portion, the second portion opening onto the inner surface 51.3.

The second part 51.2 comprises a hemicylindrical groove 51.6 of central axis perpendicular to the central axis of the bore 51.5 and of radius substantially equal to or slightly less than the radius of the rod 3.

The second jaw 52 itself also comprises a first part 52.1, or part for connecting to the screw 1, intended to be engaged on the second portion 1.2 of the screw 1 and a second part 52.2, or part for connecting to the rod 3, which laterally projects with respect to the first part and which is designed to receive one of the ends of one of the rods.

The first part 52.1 is, in this case, more or less parallelepiped in shape, with a square cross-section, identical to that of the first part 51.1 and comprises an inner surface 52.3 facing the jaw 51 (and therefore rotated inwards from the clip 50.1) and opposite an outer surface 52.4 rotated outwards from the clip 50.1. The inner surface 52.3 is a flat surface. The outer surface 52.4 is spherical cap-and convex-shaped.

The first part 52.1 of the second jaw 52 is also provided with a bore 52.5 to be engaged on the second threaded portion 1.2 of the screw 1. The bore 52.5 comprises, in this case, a first truncated cone-shaped portion and a second truncated cone-shaped portion, the first portion having a large cross-section opening onto the outer surface 52.4 and a small cross-section combined with a small cross-section of the second portion, the second portion having a large cross-section opening onto the inner surface 52.3.

The second part 52.2 is identical to the second part 51.2 and faces it, such that the grooves 51.6 and 52.6 delimit a cylindrical housing to receive the end of the rod 3.

The clip 50.1 is formed by assembling the first jaw 51 with the second jaw 52 by the pin 55. In the closed position, the grooves 51.6, 52.6 of the second parts 51.2, 52.2 of the jaws 51 and 52 form a housing of circular cross-section less than that of the ends of the rod 3 making it possible to surround an end of the rod 3 there.

The securing assembly F1 further comprises a first support sleeve 60 of the nut 80. The support sleeve is preferably distinct from the nut 80 as is represented in FIG. 2. The first support sleeve 60 delimits a through conduit to be able to be engaged on the second portion 1.2 of the screw 1. The conduit is cylindrical, in this case. The support sleeve 60 comprises, in this case, a spherical cap-shaped convex upper front surface 60.2 complementary of the lower surface of the nut 80 to bear against it, and a spherical cap-shaped concave lower front surface 60.1 complementary of the outer surface 52.4 of the first part 52.1 of the second jaw 52.

Thus, a screw 1 equipped with a securing assembly F1 according to the first configuration comprises successively engaged along the second screw portion 1.2: a clip 50.1, the first jaw 51 of which is in contact with the flange 2, and the second jaw 52 of which bears on the lower front surface 60.1 of the first support sleeve 60, the upper front surface 60.2 of said first support sleeve 60 bearing on the lower surface of the nut 80. The bore of the first and of the second jaws 51, 52 and the outer surfaces 51.4, 52.4 engaging with the upper surface of the flange 2 and the lower surface of the support sleeve 60 make it possible to create a ball-and socket joint connection around the screw.

It will be noted that the centre of the spherical cap of the outer surface 51.4 and the centre of the spherical cap of the outer surface 52.4 are combined and disposed at the centre of the small cross-sections of the first and second portions of the bore 52.5.

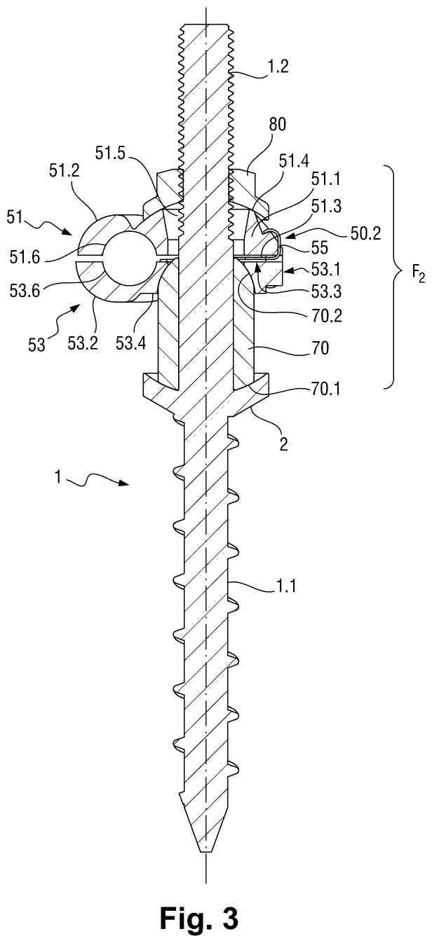

In FIG. 3, a securing assembly F2 has been represented according to a second configuration. The securing assembly F2 comprises a second support sleeve 70, a clip 50.2 and a nut 80.

The clip 50.2 is composed of two jaws 51, 53 connected by an elastically deformable pin 55. : a first jaw 51 and a third jaw 53.

The first jaw 51 is identical to that described above, but is positioned to bear against the lower surface of the nut 80. It is reminded that the lower bearing surface of the nut 80 and the upper bearing surface of the flange 2 being of identical shapes, the outer surface 51.4 can equally bear against one or the other by forming a ball-and-socket joint connection.

The third jaw 53 itself also comprises a first part 53.1, or part for connecting to the screw 1, intended to be engaged on the second portion 1.2 of the screw 1 and a second part 53.2, or part for connecting to the rod 3, which laterally projects with respect to the first part 53.1 and which is designed to receive one of the ends of one of the rods.

The first part 53.1 is, in this case, more or less parallelepiped in shape with a square cross-section, identical to that of the first part 51.1 and comprises an inner surface 53.3 facing the jaw 51 (and therefore rotated inwards from the clip 50.2) and opposite an outer surface 53.4 rotated outwards from the clip 50.1. The inner surface 53.3 is a flat surface. The spherical cap- and concave-shaped outer surface 53.4.

The first part 53.1 of the third jaw 53 is also provided with a bore 53.5 to be engaged on the second threaded portion 1.2 of the screw 1. The bore 53.5 opens downwards on the outer surface 53.4 and upwards on the inner surface 53.3. Given the concavity of the outer surface 53.4 which enters into the first part 53.1, the bore 53.5 has a minimal length.

The second part 53.2 is identical to the second part 51.2 and faces it, such that the grooves 51.6 and 53.6 delimit a cylindrical housing to receive the end of the rod 3.

The second support sleeve 70 delimits a through conduit to be able to be engaged on the second portion 1.2 of the screw 1. The conduit is cylindrical, in this case. It can be threaded or not. The second support sleeve 70 comprises, in this case, a convex- and spherical cap-shaped lower front surface 70.1 complementary of the upper face of the flange 2 and a concave-and spherical cap-shaped upper front surface 70.2 complementary of the concave outer surface 53.4 of the first part 53.1 of the third jaw 53.

Thus, a screw 1 equipped with the securing assembly F2 in the second configuration comprises, successively engaged along the second portion 1.2 of the screw 1: a second support sleeve 70, the lower front surface 70.1 of which bears against the flange 2, a clip 50.2, the third jaw 53 of which has its outer surface 53.4 bearing on the upper front surface 70.2 of the second support sleeve 70, and the first jaw 51 of which has its outer surface 51.4 bearing against the lower bearing surface of the nut 80. The bore 51.5 of the first jaw 51, as well as the outer surfaces 51.4, 53.4 engaging with the lower surface of the nut 80 and the upper front surface 70.2 of the second support sleeve 70 make it possible to create a ball-and-socket joint connection of the clip 50.2 around the screw 1.

It will be noted that the centre of the spherical cap of the outer surface 51.4 and the centre of the spherical cap of the outer surface 53.4 are combined.

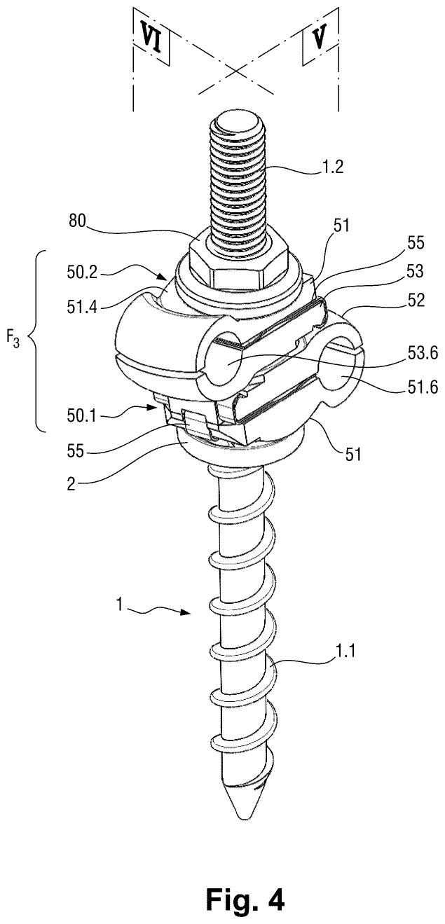

In reference to FIGS. 4 to 8, a securing assembly F3 according to a third configuration comprises two clips, a lower clip 50.1 and an upper clip 50.2. The lower clip 50.1 is identical to that of the securing assembly F1 described above and the clip 50.2 is identical to that of the securing assembly F2 described above. The outer surface 51.4 of the jaw 51 of the lower clip 50.1 bears on the upper surface of the flange 2; the outer surface 52.4 of the jaw 52 of the lower clip 50.1 bears against the outer surface 53.4 of the jaw 53 of the upper clip 50.2; the outer surface 51.4 of the jaw 51 of the upper clip 50.2 bears on the lower surface of the nut 80. Thus, a ball-and-socket joint connection of the clips 50.1 and 50.2 on the screw 1 is ensured.

Thus, a screw 1 equipped with the securing assembly F3 according to the third configuration comprises successively, a lower clip 50.1, the first jaw 51 of which has its outer surface 51.4 bears against the upper surface of the flange 2, and the second jaw 52 of which has its outer surface 52.4 bears against the outer surface 53.4 of the third jaw 53 forming with another first jaw 51, the upper clip 50.2, the outer surface 51.4 of the first jaw 51 of the upper clip 50.2 bears against the lower surface of the nut 80. The bore of the first jaws 51 of the first lower clip 50.1 and of the second upper clip 50.2, the “diabolo-shaped” bore of the second jaw 52 of the lower clip 50.1, and the complementary spherical cap-shaped surfaces in contact (the outer surface 51.4 of the upper clip 50.2 with the lower surface of the nut 80; the outer surface 51.4 of the lower clip 50.1 with the upper surface of the flange 2; the outer surface 52.4 of the lower clip 50.1 with the outer surface 53.4 of the upper clip 50.2) make it possible to create a ball-and-socket joint connection of the two clips (individually and collectively) around the screw 1.

It will be noted that the centre of the spherical cap of the two outer surfaces 51.4 are combined and disposed at the centre of the small cross-sections of the first and second truncated cone-shaped portions of the bore 52.5.

Thus, in the implantation illustrated in FIG. 1 to stabilize three adjacent vertebrae Va, Vb, Vc, the surgeon will use six screws 1, four rods 3 and six securing assemblies.

Thus, the first vertebra Va is equipped, to the left of the median sagittal plane, with a screw la provided with the securing assembly F1, and to the right of the median sagittal plane, with a screw 1′a provided with the securing assembly F2.

The second vertebra Vb is equipped, to the left of the median sagittal plane, with a screw 1′b provided with the securing assembly F3, and to the right of the median sagittal plane, with a screw 1b provided with the securing assembly F3.

The third vertebra Vc is equipped, to the left of the median sagittal plane, with a screw 1c provided with the securing assembly F2, and to the right of the median sagittal plane, with a screw 1′c provided with the securing assembly F1.

Once the screws are inserted in the vertebrae, the surgeon will position the rods 3a, 3′a, 3b, 3′b, starting with those which are located closest to the spinal column. Thus, the rod 3′a has a first end secured by the clip 50.1 of the securing assembly F1 of the screw 1′a, the second end of the rod 3′a being secured by the lower clip 50.1 of the securing assembly F3 of the screw 1′b. The rod 3b is, itself, secured to each of its ends, respectively by the lower clip 50.1 of the securing assembly F3 of the screw 1b and by the clip 50.1 of the securing assembly F1 of the screw 1c.

The rods 3a and 3′b are then respectively secured. The rod 3a is secured by a first end to the clip 50.2 of the securing assembly F2 of the screw la and by a second end to the upper clip 50.2 of the securing assembly F3 of the screw 1b. The rod 3′b is, itself, secured between the clips 50.2 of the screw 1′b and 50.2 of the screw 1′c.

The rods 3a and 3′b are secured, such that they pass respectively above the rods 3′a and 3b, the rods crossing one another substantially over the median sagittal plane. The rods 3′a and 3a thus stabilize the vertebrae Va and Vb against one another, the rods 3′b and 3b stabilize the vertebrae Vb and Vc against one another.

The surgeon will know how to adjust the positioning of the rods, thanks to the rotation of the clips around each screw 1 and to the ball-and-socket joint connections of each of the screws 1, made possible by the bores of the jaws of the clips. This therefore enables an extremely accurate adjustment in the three dimensions of the space: in height by the positioning of the clips along the second screw portions, in length by the rotation of the clips around the screws and inclined thanks to the ball-and-socket joint connections, while preserving a very high compactness of the device according to the invention. Once the rods are in their optimal position, the surgeon will secure the device by clamping the nut 80 on each screw and cut the second portion of each screw at the breaking piece 1.4 (represented in FIG. 2).

For other pathologies, only two adjacent vertebrae can be stabilized. In this case, the assembly used comprises two pairs of screws (1a, 1b and 1′a, 1′b). The screws 1′a, 1′b are equipped with the securing assembly F1, while the screws 1a and 1b are equipped with the securing assembly F2. The screws 1a and 1′a are inserted on either side of the first vertebra, respectively to the left and to the right of the median sagittal plane, the screws 1b and 1′b are inserted on either side of the second vertebra, respectively to the right and to the left of the median sagittal plane. A first rod 3 is then secured between the screws 1′a and 1′b, a second rod 3 between the screws 1a and 1b.

Thus, the device according to the invention used to stabilize the vertebrae is easy to implement and is unobtrusive. The number of screws of each device is established according to the number of vertebrae to be fitted. The use of the device according to the invention enables an optimal and accurate adjustment of the position of the rods regarding the pathology of the patient and their morphology. Furthermore, it is absolutely possible to easily modify this adjustment as the pathology evolves, simply by modifying the respective position of the clips, by changing the securing assembly engaged on the screw or by changing the rods, if they are no longer suitable, without considering a long operation for the patient.

This great accuracy in adjusting the positioning of the rods, choosing their length and their adjustment on the jaws of the clips makes it a bespoke system, adapted to each patient, although comprising securing elements which are themselves, of standard manufacture.

To also facilitate the implementation of the device, each element of it (screws, securing assemblies: jaws, support sleeves, nuts and rods) can form the subject of a marking or colour, making it possible to both rapidly identify the element, and also trace it from manufacture to the implementation in the body of the patient. The marking can be done by any biocompatible means known as such.

Naturally, the invention is not limited to the embodiments described, but includes any variant entering into the field of the invention, such as defined by the claims.

In particular, the instrumented vertebrae are, in this case, adjacent, but they might not be. The length of the rods must thus be simply adapted, according to the distance between the two vertebrae to be instrumented.

In this case, the rods are thus secured between the vertebrae in a crossed manner, but the device according to the invention can absolutely enable the securing of the rods parallel to the median sagittal plane. Furthermore, although in this case, one single securing mode is presented (securing with crossed rods), it can be provided, according to the pathology of the patient, to use the assemblies of devices according to the invention to instrument certain vertebrae in a crossed manner, and other vertebrae in parallel.

The rod has, in this case, a circular cross-section, but it could have an oval, triangular, rectangular, polygonal cross-section, etc.

The rod has, in this case, a constant cross-section, but it could have a cross-section which is narrower at the ends than at the centre, or vice versa, wider at the ends than at the centre.

The rods have, in this case, a predefined length, but they could be custom cut to the desired length.

The rods could be rectilinear instead of being curved.

The screws are solid, in this case, but they could be hollow to enable the securing or the passage of a pin to facilitate a percutaneous surgery.

The jaws have, in this case, a substantially parallelepiped shape, but can have any shape, for example, cylindrical, polyhedral, or ovoid, etc. The groove of the second part can have an angular or polygonal cross-section, such that once the two jaws of the clip are closed, the second parts of the jaws forms a channel of square or polygonal cross-section.

The device could comprise nuts integrating support sleeves 60 in one single part, and/or screws integrating support sleeves 70 in one single part, and/or screws and nuts such as described, in this case.

The securing assemblies F1 and F2 comprise, in this case, a support sleeve, but this could be replaced by one or more foldable washers according to the height sought, the first and the last washer taking the shape of the lower and upper front surfaces of the support sleeve, or the support sleeve could be completely absent from the securing assembly, if the anatomy of the patient makes it possible.

In particular, in this case, the device of the invention applies to human beings, but it can be adapted to any vertebra, in particular pets, like horses or dogs. Naturally, a person skilled in the art will know how to adapt the dimensions of the elements of the device, according, in particular, to the length and the diameter of the screws and of the rods, and consequently, the dimensions of the clips and, of the support sleeves and of the nut.

1. Vertebral stabilization device, comprising:
at least two screws, each having a first threaded portion to be engaged in a vertebra and a second threaded portion separated from the first threaded portion by a flange;
at least one first connection rod having two opposite ends to connect the two threaded portions of the screws to one another; and
at least one first securing assembly and one second securing assembly to secure the ends of the rod to the screws, each securing assembly comprising at least one clip, which is able to be engaged on the second threaded portion of one of the screws and arranged to clamp an end of the rod, and a nut which has a lower bearing surface and which is designed to be screwed on the second threaded portion by clamping the clip against an upper surface of the flange of the screw, each securing assembly being arranged to create a ball-and-socket joint connection between the clip and the second threaded portion.

2. Device according to claim 1, wherein the lower bearing surface of the nut and the upper bearing surface of the flange are spherical cap-shaped concave surfaces which are identical to one another.

3. Device according to claim 1, wherein the clip at least of the first securing assembly comprises a first jaw having a complementary spherical cap-shaped convex outer surface to bear on the upper surface of the flange, and a second jaw having a spherical cap-shaped convex outer surface, the first securing assembly comprising a first support sleeve of the nut having a complementary lower front surface to bear against the spherical cap-shaped convex outer surface of the second jaw.

4. Device according to claim 3, wherein the first support sleeve is distinct from the nut.

5. Device according to claim 4, wherein the first support sleeve comprises a spherical cap-shaped convex upper front surface complementary of the lower surface of the nut to bear against it.

6. Device according to claim 1, wherein the clip at least of the first securing assembly comprises a first jaw having a complementary spherical cap-shaped convex outer surface to bear against the lower surface of the nut and a third jaw having a spherical cap-shaped concave outer surface, the first securing assembly comprising a second support sleeve having a complementary upper front surface to bear on the spherical cap-shaped concave outer surface of the third jaw.

7. Device according to claim 6, wherein the second support sleeve has a complementary spherical cap-shaped concave lower front surface to bear on the upper surface of the flange.

8. Device according to claim 1, wherein the second assembly comprises two clips, namely a lower clip and an upper clip, the lower clip comprising a first jaw having a complementary spherical cap-shaped convex outer surface to bear on the upper surface of the flange, and a second jaw having a spherical cap-shaped convex outer surface, the upper clip comprising a first jaw having a complementary spherical cap-shaped convex outer surface to bear against the lower surface of the nut and a third jaw having a spherical cap-shaped concave outer surface complementary of the spherical cap-shaped convex outer surface of the second jaw to bear on it.

9. Device according to claim 3, wherein each clip comprises an elastically deformable portion connecting the jaws, such that these are movable against one another between an open position and a closed position.

10. Device according to claim 3, wherein the first jaw is provided with a bore to be engaged on the second threaded portion of the screw, the bore comprising a first truncated cone-shaped portion and a second cylindrical portion, the first portion having a large cross-section opening onto the outer surface of the first jaw and a small cross-section combined with a cylindrical cross-section of the second portion, the second portion opening onto the inner surface of the first jaw.

11. Device according to claim 3, wherein the second jaw is provided with a bore to be engaged on the second threaded portion of the screw, the bore comprising a first truncated cone-shaped portion and a second truncated cone-shaped portion, the first portion having a large cross-section opening onto the outer surface of the second jaw and a small cross-section combined with a small cross-section of the second portion, the second portion having a large cross-section opening onto the inner surface of the second jaw, the common centre of rotation being disposed against the small cross-sections of the first and second portions.

12. Device according to claim 1, wherein the flange is removable.

13. Device according to claim 1, wherein the rod has a constant cross-section over its entire length.

14. Device according to claim 1, wherein the rod is curved.

15. Device according to claim 1, wherein the second threaded portion of each screw is provided with an end portion designed to enable a rotating connection with a tool for screwing the screw into the vertebra, and a breaking piece to be able to separate the end portion from the second threaded portion by breaking.
